(19) United States
(12) Patent Application Publication
(10) Pub. No.: US 2016/0325112 A1
(43) Pub. Date: Nov. 10, 2016

(54) PHOTON UPCONVERTING IMPLANTABLE MEDICAL DEVICES

(71) Applicant: Stephen Eliot Zweig, Los Gatos, CA (US)

(72) Inventor: Stephen Eliot Zweig, Los Gatos, CA (US)

(21) Appl. No.: 15/154,933

(22) Filed: May 13, 2016

Related U.S. Application Data
(63) Continuation-in-part of application No. 12/871,899, filed on Aug. 30, 2010, now abandoned.
(60) Provisional application No. 61/242,025, filed on Sep. 14, 2009.
(60) Provisional application No. 61/238,201, filed on Aug. 30, 2009.

(51) Int. Cl. A61N 5/06; A61M 5/142; A61F 2/82; A61M 5/00; A61L 31/02; A61N 5/10

(52) U.S. Cl. CPC A61N 5/062; A61L 31/022; A61N 5/0624; A61N 5/1002; A61F 2/82; A61M 5/007; A61M 5/14276; A61N 2005/0602; A61N 2005/0659; A61N 2005/0663; A61N 2005/0661; A61N 2005/1024; A61F 2002/825; A61F 2250/0067

(57) ABSTRACT

An active material, suitable for implantable medical devices. The active material is a mixture, composite, or aggregate of upconverting particles and photocatalytic particles embedded in an at least infrared light conducting and gas permeable support material. The upconverting particles convert longer wavelength light into shorter wavelength light, which in turn powers the photocatalytic materials to produce cytotoxic chemicals. When embedded into an implantable medical device in a desired body location, and exposed to red or infrared light, this active material can produce cytotoxic chemicals that in turn exert a cytotoxic effect on unwanted cells, such as cancer or microbial cells, as well as perform other functions.

### CROSS REFERENCE TO RELATED APPLICATIONS

This application is a continuation in part of U.S. patent application Ser. No. 12/871,899, “PHOTON UPCONVERTING IMPLANTABLE MEDICAL DEVICES”, filed Aug. 30, 2010, inventor Stephen Eliot Zweig. Application Ser. No. 12/871,899 claimed the priority benefit of provisional patent application No. 61/238,201, “Photon upconverting implantable medical devices”, filed Aug. 30, 2009, and provisional patent application No. 61/242,025, filed Sep. 14, 2009, Stephen Eliot Zweig inventor; the contents of all of these applications are hereby incorporated herein by reference in their entirety.

### BACKGROUND OF THE INVENTION

1. Field of the Invention

The invention covers certain methods and devices designed to improve the performance of implanted medical devices. The invention also covers certain improvements in the field of drug administration technology and drug administration devices. The invention also covers certain improvements in the field of radiation and light administration therapy.

2. Description of the Related Art

Stents, catheters, and other related medical devices have proven useful for the treatment and prevention of preventing coronary artery disease, and other disorders.

Stents are commonly introduced to the inside of a diseased artery or other body lumen using a catheter. The stent is normally inflated inside the artery or body lumen, and the catheter removed. The stent then acts to hold the artery or body lumen open, and can also help prevent subsequent reclosure (stenosis).

Catheters are used for a wide variety of medical purposes. Some purposes, such as cardiac treatment, require only very short-term use in the body. Other purposes, such as administering blood, drugs, and nutrition intravenously, as well as draining urine from the urethra, require that the catheters be implanted into the body for extended periods of time.

One common problem with stents is unwanted cell proliferation and hyperplasia. Often the inner wall of the artery is damaged, either by the disease, or alternatively by irritation induced during the stent introduction or stent material itself. As a result, the body's natural defense mechanisms act to cover the stent material by a process of cell proliferation. If unchecked, the resulting hyperplasia and cell overgrowth in the stent area can act to narrow or close the stent, resulting in blocked circulation, and rendering the stent ineffective.

Numerous prior art attempts have been made to prevent stent closure. These methods include making stents of radioactive material, and embedding various types of cell proliferation inhibiting or cytotoxic drugs into the stent material itself. Radioactive stents have presented safety problems, however, and have not been totally effective. Similarly drug eluting stents, although promising, have not been totally effective either. Stents are small, and only a small amount of drug can be embedded or associated with the stent. Eventually this small amount of drug runs out, and thus the stent no longer can prevent unwanted cell proliferation.

Bernstein, in US patent application 2009/0130169, proposed employing phosophors that converted electromagnetic radiation to cytotoxic ultraviolet UVC radiation (e.g. the 250 to 260 nm range) into stents, and using the damaging effect of UVC radiation on proliferating cells to inhibit hyperplasia. However the efficiency of upconverting light to such short wavelengths will generally be low with most known materials. Additional problems are that these short wavelengths are intensely absorbed by nucleic acids and other materials in the body, and thus their penetration range will be extremely short. Finally, the direct killing power of UVC light is not overly high, and thus large amounts of UVC light will need to be produced in order to produce the desired cytotoxic effect.

Thus methods to improve stent performance, particularly to prevent unwanted cell proliferation, stent closure and restenosis, are desirable.

For catheters, a different type of problem, infection, presents itself. Many catheters span the junction between the non-sterile environment outside the body, and the ideally sterile environment inside the body, and the catheter-body junction presents a location where bacteria, fungi, and other pathogens may enter the body.

Previous workers, such as Yao et. al., “Self-Sterilization using Silicone Catheters Coated with Ag and TiO₂ Nanocomposite thin film”, Journal of Biomedical Materials Research Part B: Applied Biomaterials Volume 85B Issue 2, Pages 453-460 (2007) attempted to use the known sterilization capabilities of Titanium dioxide (TiO₂) photocatalysis, which is activated by exposure to UV light, to reduce the danger caused by bacterial colonization of such catheters. However, as they discuss, “it is impossible for the photocatalyst to work in dark conditions such as inside the human body” (p 454 column 1, first paragraph).

Thus methods to generally improve the on-demand cytotoxic or antimicrobial performance of a range of various types of implanted medical materials are also desirable.

### BRIEF SUMMARY OF THE INVENTION

In one embodiment, the invention may be an active material comprising at least one non-soluble active material formed from upconverting particles and photocatalytic particles mixed together in at least one different non-toxic, non-soluble, gas permeable biocompatible particle support material so as to form a compound or aggregate. This active material is configured so that when it is exposed to red or infrared light, the upconverting particles emit shorter wavelength light which is subsequently absorbed by the photocatalytic particles, which in turn emit cytotoxic chemicals or perform other chemical reactions in response to said shorter wavelength light.

This active material has many uses, and is particularly useful for implantable medical devices for a number of interesting applications. In some embodiments, the active material may be used in a device, system, and method to preventing occlusion of arteries and veins by means of a stent that contains a photon (light) upconverting material.

Note that for the purposes of this disclosure, “upconverting material” is a material that is capable of absorbing multiple lower energy (longer wavelength) photons, upconverting these lower energy photons to higher energy photons, and then emitting these higher energy (shorter wavelength) photons.

In another embodiment, the invention's active material may be used in improved device, system, and method for preventing infection in the body, by means of an implanted medical device, such as a catheter, that contains both an upconverting material and a light activated photocatalyst, such as TiO₂ or other material that has antimicrobial or cytotoxic properties upon exposure to light.

In other aspects of the invention's active material include other types of improved implantable medical devices incorporating the active material. In addition to the benefits of implantable stents that incorporate the active material, a number of other implantable devices, including, but not limited to orthopedic medical implants, spinal implants, brachytherapy seeds or pellets, and drug administration implants may also benefit from this approach.

In yet another aspect of the invention, the specificity of drug administration devices may be further enhanced by strategic use of active material containing medical implants constructed using at least some upconverting materials.

One important principle of the invention is to utilize the ability of an upconverting material to convert lower energy (longer wavelength) illuminating photons into higher energy (shorter wavelength) photons that are subsequently emitted by the upconverting material.

Here, the advantages of upconverting materials can be enhanced by taking advantage of the fact that the dominant color absorbing material in the human body is hemoglobin (predominantly oxyhemoglobin). Hemoglobin has very little light absorption in the wavelengths below about 600 nm. Thus, to a first approximation, the human body may be viewed as being semi-transparent or semi-translucent at longer wavelengths, typically considered to be in the near infrared (near IR) region. As a result, such longer wavelengths can travel for a considerable distance in the human body, such that illuminating light sources can be placed some distance away from the illumination target (indeed the light sources may even be placed outside the body in some cases), and the illumination light can travel a significant distance through the human body to reach the upconverting material.

By contrast, the higher energy photons emitted by the upconverting material will often be at a wavelength that is highly absorbed by hemoglobin, and thus the higher energy photons will travel only a very short distance. Because higher energy photons are generally more effective at stimulating a wide variety of different chemical reactions, this combination of short distance (localization) and higher ability to induce chemical reactions can be utilized for a wide variety of therapeutic applications.

In particular, there are a number of upconverting materials that can take illumination light at wavelengths greater than about 600 nm, and in turn upconvert the light and produce either shorter wavelength blue light at wavelengths of around 475 nm or shorter, violet light at wavelengths of 400 or shorter, or ultraviolet light at wavelengths between 400 nm and 320 nm. Even shorter wavelength violet light, Ultraviolet B light, in the 320-280 nm region has been reported.

As previously discussed, the shorter wavelength light produced by the upconverting material is more energetic than the longer wavelength light used to excite the upconverting material, and as a result, the shorter wavelength light may be employed to do a number of useful chemical reactions. In one use, the shorter wavelength light may be used to kill or damage nearby cells directly.

In an alternative and generally preferred use, the shorter wavelength light may be used to activate a light activated light activated photocatalyst, such as TiO₂, and exhibit desired cytotoxic or antimicrobial effects indirectly. This light activated photocatalyst may also be used to conduct other useful chemical reactions, such as to convert inactive forms of drugs to active forms of drugs, and the like.

In yet another alternative use, the shorter wavelength light, in conjunction with a suitable photocatalyst, may be used to convert a non-target molecule to a target-molecule, and the target molecule in turn may be recognized by various receptor molecules, which in turn may deliver various payloads, such as cytotoxic agents, immunological agents, regulatory agents, and the like to cells and tissues nearby the implanted upconverting medical device. In yet another alternative use, the localized and shorter wavelength light, in conjunction with the photocatalyst, may be used to enhance the efficiency of various diagnostic procedures and methods.

Thus by suitable use of upconverting implanted medical devices, longer wavelength incident illumination, and one or more optional photocatalysts, light sensitive drugs and drug combinations, the property of the tissue surrounding the implant may be modified in useful ways.

In the case of a stent, unwanted cell hyperplasia and other proliferation that puts the stent at risk for premature closing can be eliminated. For an orthopedic implant, a suitable immune cell suppressant function might be employed to reduce the amount of inflammation or arthritis in the region. For an implant designed to combat a tumor or unwanted growth, such as the various “seeds” or “pellets” used in brachytherapy to treat diseases such as prostate hyperplasia or cancer, such methods can help direct cytotoxic and inflammatory reducing agents to desired portions of the body.

Thus some embodiments, the invention may be a macroscopic sized implantable medical device comprising upconverting particles and photocatalytic particles, in which the device comprises a particle support material comprised of a non-upconverting material and non-photocatalytic material, herein termed a “particle support material”. Here a mixture of the upconverting particles and photocatalytic particles is embedded in the non-upconverting particle support material, thereby creating “active material”.

The macroscopic sized implantable device will typically have a longest dimension of at least one millimeter (e.g. macroscopic sized seeds intended to remain where they are originally implanted), and indeed may have a longest dimension greater than 1 centimeter (e.g. for stent applications), and in some embodiments (such as catheter applications) may have a longest dimension of 10 centimeters or more. The macroscopic sized implantable device will often have its shape controlled by either the shape of the non-upconverting particle support material, or by shape of yet another material that holds the active material. Typically the shape and size of the device will be designed so that the macroscopic sized implantable device remains in the location of the body where it was originally implanted.

### BRIEF DESCRIPTION OF THE DRAWINGS

FIG. 1 shows a stent configuration useful for holding light upconverting particles, and pressing these light upconverting particles against the inner wall of an artery or vein.

FIG. 2 shows a detail of how light upconverting particles operate.

FIG. 3 shows the light absorption spectra of oxyhemoglobin in the visible and near infrared regions.

FIG. 4 shows examples of various ways to illuminate light upconverting implantable devices such as stents.

FIG. 5 shows how a pellet containing upconverting material may be used, in conjunction with light activated drugs, to treat a tumor or other diseased portion of tissue.

FIG. 6 shows use of small (millimeter size) or microscopic (size of 1000 microns or less) soluble upconverting particles for diagnosis or treatment.

FIG. 7 shows a medical catheter, such as a Broviac® catheter, or other catheter, comprised of both upconverting particles and particles of a photocatalytic antibacterial material such as particles of titanium dioxide, and surrounded by a biofilm of an undesired pathogenic bacteria. By illuminating the catheter with 600-800 nm light, which penetrates deeply into the body, the upconverting particles produce shorter wavelength light, which activates the photoactivated antibacterial material. The photoactivated antibacterial material in turn kills the various undesired pathogenic bacteria in the biofilm.

FIG. 8 shows one possible mechanism by which a particle of an upconverting material can convert infrared light to shorter wavelength light, activating a photocatalyst, and resulting in destruction of microbes.

FIG. 9 shows a mixture, compound, or aggregate material of various upconverting and photocatalytic particles dispersed inside of a non-toxic, non-soluble biocompatible particle support material, thus forming active material. In a preferred embodiment, this support material conducts at least infrared light, and allows cytotoxic chemicals generated by the various photocatalytic particles inside of the active material to exit the active material.

### DETAILED DESCRIPTION OF THE INVENTION

Although throughout this disclosure, occasionally stents and catheters will be used as specific examples of implantable medical devices that can be improved by the use of the disclosed active material. However it should be understood that these examples are simply provided for ease of conveying the basic concept, and are not intended to be limiting in any way.

Stent applications:

Some modern stents, such as the stent design of Stanley (U.S. Pat. Nos. 6,565,065 and 7,169,179) (Conor Medsystems) are formed from a deformable material, such as Nitinol (nickel-titanium), other metal or alloy, or other deformable material, and often containing a plurality of small holes. The prior art for such small holes, exemplified by U.S. Pat. No. 7,169,179 is to fill the small holes with therapeutic agents (drugs), often packaged with time release barriers and the like. These therapeutic drugs can then be used to prevent restenosis, thrombosis, or other subsequent unwanted biological response that could diminish the effectiveness of the stent. The drawback of this time release drug concept, however is that the volume of the plurality of small holes is very small, and eventually most or all of the drug will elute.

However consider if the small holes are instead filled with particles of an active material, which can often be packaged in the form of a relatively biologically inert glass, then the ability of the upconverting material to convert longer wavelength incident light to shorter wavelength emitted light is effectively infinite. Neglecting upconverting material deterioration, as long as there is a source of illumination light, the upconverting material will produce shorter wavelength photons. These shorter wavelength photons can be produced for many years, if necessary, in contrast to drugs, which often become exhausted within weeks or months.

Further, if the shorter wavelength photons are used in conjunction with appropriate light sensitive therapeutic drugs or other useful photosensitized agents (for example photosensitized agents such as titanium dioxide (TiO₂), cells in the vicinity of the implanted stent can be modified in a variety of ways. These cells can be damaged or destroyed by light triggered cytotoxic drugs, be inhibited by appropriate light triggered target molecules, or be induced to enter other desired therapeutic states depending upon the treatment modality desired. Indeed, a stent or other therapeutic device could be implanted, treated in conjunction with a first available drug (such as a psorlen) or photosensitized agent for a number of years, and then used in conjunction with a new light sensitive drug or photosensitized agent, years later, that may not have even been available at the time that the upconverting stent was originally implanted.

If the stent is constructed with a mixture of upconverting material and photoactivated catalysts (again using TiO₂ as a specific example), the photoactivated catalyst may exert its local cytotoxic effect by, for example, producing strong oxidants, such as hydroxyl radicals (OH) and superoxide radicals (O⁻₂), effectively a cytotoxic gas. Thus, for example, when the upconverting material is exposed to deeply tissue penetrating infrared light, such as light in the 600-800 nm region, the upconverting material produces shorter wavelength light that activates the photocatalyst. The photoactivated catalyst produces cytotoxic agents, such as strongly oxidizing radicals, which in turn can destroy unwanted cell hyperplasia and other proliferation causing unwanted restenosis of the stent.

If the upconverting material (particles), the photo activated catalysts (particles) are mixed into a substantially red or infrared conducting support material that is also at least permeable to the cytotoxic gasses, then the effectiveness of the system would be still further enhanced. Thus in the preferred embodiment, the holes in the stent would be filled with active material.

Note that the cytotoxic effect of such cytotoxic agents will be dose dependent (i.e. stronger concentrations of cytotoxic agents will be more effective at killing cells, while weaker concentrations of cytotoxic agents will be less effective at killing cells). When the cytotoxic agents are coming from a macroscopic sized medical device that is located in a fixed position, then the cytotoxic effect of the cytotoxic agents emitted by the fixed position macroscopic sized medical device will fall off sharply as a function of the distance away from the fixed position macroscopic sized medical device. The exact function will vary according the size and shape of the macroscopic sized medical device, with smaller, more “point” sized devices having more of a 1/distance² fall off, and larger medical devices having more of a gentler 1/distance type fall off. Other effects that can modify these cytotoxic effects include the lifetime of the cytotoxic agent, blood or other fluid flows in the region of the implanted medical device, and the like.

This fall off with distance effect is generally desirable, as often it will be desired to only kill off unwanted cells within a short range of the implanted macroscopic sized medical device. Thus for stent and catheter applications, often it will be desirable to configure the active material and amounts and times of red or infrared light exposure so that only cells located 1 millimeter or less away from the implanted macroscopic sized medical device are killed, and cells at greater distances are spared. By contrast, for some implanted seed applications, where the seeds may be implanted in solid tumors, it may be desirable to configure the active material and amounts and times of red or infrared light exposure so that unwanted tumor cells greater than 1 millimeter away from the implanted seed are killed. Even here, however, there is an upper limit and generally it will be undesirable for such implantable seeds to be configured with a cytotoxic radius of greater than 1 centimeter, since here too many desired (healthy) cells will be damaged.

Although in this disclosure will mainly focus on utility of the active material to produce precisely localized amounts of cytotoxic chemicals, the active material disclosed herein may also be used for other applications and purposes as well.

In other embodiments, the active material can also be used for diagnostic purposes. One way is by injecting a suitable dye into circulation. If the active material is part of a stent or other device that is freely exposed to the blood circulation, photocatalytic materials may be able to interact with the injected dye and cause the dye to fluoresce or emit some other signal at a third wavelength, that can then be detected. However if the stent is overgrown, the ability of the photocatalytic material to interact with the injected dye will be diminished due to the interfering effect of the overgrowing cells, and this effect might also be detected.

Returning to the upconverting stent embodiment, as one example, the upconverting stent can be produced by filling the plurality of holes from a Nitinol Stanley (U.S. Pat. Nos. 6,565,065/7,169,179) stent design with an active material formed from an underlying plastic, glass, or ceramic based support material. The resulting stent would essentially retain its normal characteristics of being capable of being collapsed, employed by a suitable catheter system, and then expanded to fill the appropriate artery or vein. Here the deformable Nitinol stent structure (100) would be termed the “active material support material”. The upconverting particles and photocatalytic particles are embedded in the plastic glass or ceramic material, here termed “particle support material”, and this would form “active material”. The “active material” can then be used to fill the holes in the stent “active material support material” as previously discussed.

In some embodiments, the invention may be an implantable medical device comprising at least one non-soluble active material formed from a mixture of separate upconverting particles and separate photocatalytic particles embedded together in at least one different non-toxic, non-soluble biocompatible particle support material that holds the active material in an active material shape. The device may optionally further comprise a non-soluble biocompatible active material support material disposed to hold the active material. As before, when this active material is exposed to red or infrared light, the upconverting particles emit shorter wavelength light which is subsequently absorbed by the photocatalytic particles, which in turn emit cytotoxic chemicals in response to the shorter wavelength light. Typically the medical device will have a device shape that is determined by either the shape of the active material, and/or the shape of any active material support materials that have been disposed to hold this active material.

The device size and shape will be such that the device remains where originally implanted. As previously discussed, the device operates by localized destruction, relative to a location of the device, of unwanted living cells surrounding the active material. This localized destruction is caused by cytotoxic chemicals that are emitted by the active material in response to an interaction of red or infrared light with the active material.

Regarding the particle support materials

The particle support material serves various functions. In addition to securely holding the upconverting particles and the photocatalytic particles, the particle support material and shape of the particle support material should ideally be configured to be consistent with the overall function of the medical device. In particular, the need to allow red or infrared light to reach the upconverting particles, the need for shorter wavelength light to travel from the upconverting particles to the photocatalytic particles, and finally the need to allow for both the formation of cytotoxic chemicals, and allowing the cytotoxic chemicals to exit the medical device are all considerations.

Although Ohko et. al., “Self-Sterilizing and Self-Cleaning of Silicone Catheters Coated with TiO₂ Photocatalyst Thin Films: A Preclinical World”, J. Biomed. Matter Res. 2001; 58(1):97-101 taught that it was possible to absorb titanium dioxide particles to the surface of a silicon substrate (such as a silicon catheter); such particle attachment methods are generally less preferable. This is because such particles have a tendency to leach from the surface, and/or to gradually be engulfed by leucocytes (e.g. macrophages, neutrophils, monocytes), which are well known to interact (and be activated by) such particles. This can lead to potential inflammation and loss of biocompatibility, as well as loss of the desirable characteristics of the particles themselves.

Ideally the various particles should be embedded into the basic structure of the particle support material in an effectively irreversible manner so as to be resistant to both leaching and attack by macrophages or other body processes. Here, it may be useful to employ at least somewhat transparent particle support materials, and mix or otherwise create a compound or aggregate of the particle support materials and the photocatalytic particles and the upconverting particles so that at least the vast majority of the various particles are no longer exposed to the surface of the support material. This renders at least the vast majority of the various particles immune from leaching and macrophage attack.

Another consideration, however is that the particle support material should also be compatible with both the production and release of cytotoxic chemicals. Here, use of certain transparent or semi-transparent biocompatible porous materials may be useful, such as polyethylene, polycarbonate, polyetheretherketone, porous plastics, gas permeable materials, sintered materials, various porous ceramics, and the like.

Ideally the pore size distribution will be such as to not admit macrophages or other body cells, while at the same time allowing for cytotoxic chemicals to diffuse away from the particle support material and into the nearby region where unwanted cells can then be destroyed. Suitable ceramics are taught by Heide, U.S. Pat. No. 3,899,556. Other suitable materials include various oxygen permeable materials used in contact lenses, such as rigid gas permeable materials, silicone hydrogels and other oxygen permeable materials.

In a preferred embodiment, the active material, although it need not be totally rigid, should at least substantially solid and rigid enough to maintain a defined shape in an aqueous environment at around 37° Centigrade. Here the rigidity should be, on the low end, at least roughly that of a soft contact lens, which is able to maintain its overall shape (even with some bending) so long as the lens is sufficiently hydrated and at around body temperature.

Thus in a preferred embodiment, the active material is a mixture of upconverting particles, photocatalytic particles, and a cytotoxic permeable particle support material. This particle support material will also preferably be light conducting, at least in the red and infrared regions of the spectrum, and optionally also in the blue and ultraviolet regions of the spectrum as well.

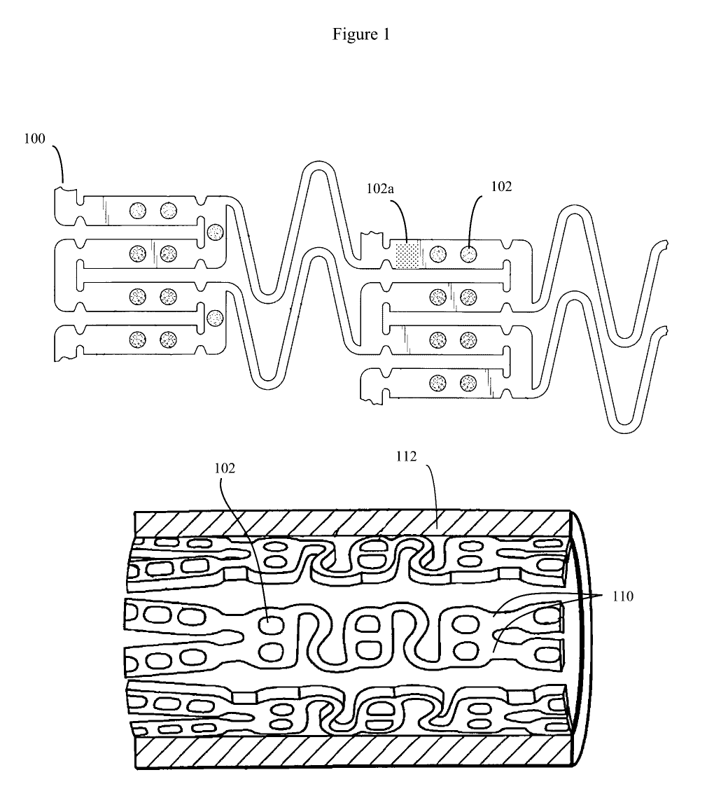

One type of device configuration is shown in FIG. 1. FIG. 1 (100) shows the stent in a flattened configuration, with a plurality of holes (102) in the (usually metal) biocompatible active material support material. Here, these holes will be filled with the previously discussed active material, which often will be formed from a mixture of upconverting particles, photocatalytic particles and particle support material. This active material may be disposed in the form of small plastic, porous glass, or ceramic disks, squares, or other shaped particles. In an alternative configuration, the stent surface may be covered a polymer (here the polymer serves as the support material), and small granules of the upconverting material (particles) and optionally also the cytotoxic material (particles) may be embedded or mixed in the polymer, thus forming the active material. In this alternative configuration (not shown), small holes (102) need not be present, and instead a coating (102a) may be present. FIG. 1 (110) shows the stent in the normal deployed configuration, pressed up against the inner wall (112) of an artery, vein, or other body lumen.

Thus when properly configured to fit into an artery or vein, the stent presses the upconverting material next to the walls of the body lumen.

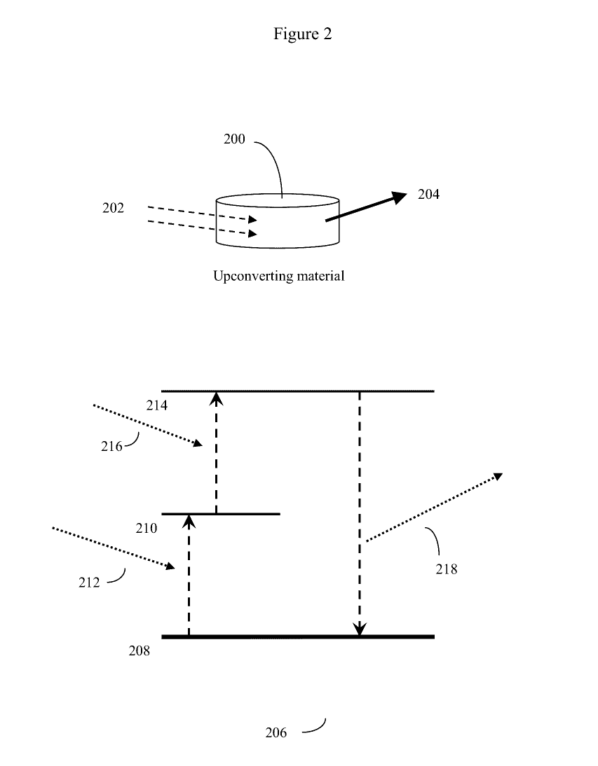

FIG. 2 shows how upconverting material operates. This upconverting material, when embedded in small disks or particles of active material (a mixture of upconverting particles, photocatalytic particles and a suitable support material), which in some embodiments be placed into the small holes (102) or used to coat the stent.

As previously discussed, on a quantum level usually the upconverting material (200) will absorb multiple (two or more) low energy photons (i.e. infrared light) (202), and emit a high energy upconverted photon (often an ultraviolet light photon) (204) as a result. On a macroscopic level, of course, the effect is essentially continuous, and the upconverting disk or particle will appear to glow at a higher wavelength when illuminated with lower wavelength incident light.

The energy level diagram (206) shows the same thing from a quantum mechanics standpoint. Here an electron at a lower energy level (208) is promoted to an intermediate energy level (210) by a first long wavelength photon (212). The electron is then promoted to a higher energy level (214) by a second long wavelength photon (216). The electron then decays back to the original energy level (208) emitting a high energy short wavelength photon (218).

A large number of different upconverting materials have been identified, and the list provided later in this document is by no means exhaustive. In general, the upconverting material should be selected to ideally accept photons that can travel a fair distance through the body (in a preferred mode, in the 600 to roughly 800 nm region), upconvert the light (with as high a quantum efficiency as feasible) and emit the light at a shorter wavelength designed to be suitable for the therapeutic application contemplated. Often this emitted light will be at a wavelength of 500 nm or less. The upconverting material will usually be designed to be sable at body temperatures, and being capable of being at least embedded in a carrier or support that is non-toxic and biocompatible enough as to be suited for that particular medical application.

Examples of Upconverting Materials:

Paz-Puj alt et. al, U.S. Pat. No. 5,786,102, incorporated herein by reference, taught a amorphous upconversion phosphor comprising barium fluoride a combination of rare-earth fluorides including yttrium and lanthanum and dopants, and a waveguide thereof on a substrate selected to have a refractive index lower than a thin film of the phosphor material or any other substrate with an appropriate buffer layer of lower refractive index that the film wherein infrared radiation and visible light are converted to ultraviolet and visible light. The amorphous upconversion phosphor is deposited at temperatures low enough to permit integration into semiconductor materials.

Chen, in U.S. Pat. No. 7,008,559, incorporated herein by reference, taught manganese doped upconversion luminescent nanoparticles.

Daqin Chen, Yuansheng Wang, Yunlong Yu, Feng Liu, and Ping Huang, “Sensitized thulium ultraviolet upconversion luminescence in Tm³⁺/Yb³⁺/Nd³⁺ triply doped nanoglass ceramics,” Opt. Lett. 32, 3068-3070 (2007) describe intense four- and five-photon ultraviolet upconversion processes through sensitization of Tm3+ ions in transparent SiO2-Al2O3-NaF-YF3 glass ceramics triply doped with Tm3+/Yb3+/Nd3+ under 796 nm excitation were investigated. Judd-Ofelt analyses evidenced the incorporation of rare-earth ions into the precipitated β-YF3 nanocrystals. In contrast with the triply doped one, no ultraviolet upconversion luminescence was observed in the Tm3+/Nd3+ codoped glass ceramic, indicating that Yb3+ acts as bridging ions to enhance the energy transfer efficiency between Nd3+ and Tm3+.

Daqin Chen, Yuansheng Wang, Yunlong Yu, and Ping Huang Intense ultraviolet upconversion luminescence from Tm3+/Yb3+:beta-YF3 nanocrystals embedded glass ceramic Appl. Phys. Lett. 91, 051920 (2007); doi:10.1063/1.2767988.

Chen found that the infrared to ultraviolet upconversion emissions of Tm3+1I6→3F4 (346 nm) and 1D2→3H6 (362 nm) transitions, originating from the five- and four-photon upconversion processes, respectively, were observed in the Tm3+/Yb3+ codoped precursor glass and glass ceramic containing beta-YF3 nanocrystals. The ultraviolet luminescence of the glass ceramic is 30 times stronger than that of the precursor glass, which could be attributed to the decreased probability of the 3F2→3F4 transition and the increased cross relaxation of 3F2+3H4→3H6+1D2 resulted from the partition of rare earth ions into nanocrystals.

Light Sources:

As previously discussed, in the case of a stent, or other medical implant, the IR to UV upconverting material can often be illuminated by an infrared (IR) or red light source from outside of the artery, or even from some distance away. If the light source is sufficiently powerful and at an optimized wavelength, the illumination light may even come from outside of the body.

The reason why the IR light source can be located some distance away from the upconverting stent or other medical device containing upconverting material is that the main light absorbing molecule in the human body is hemoglobin, and hemoglobin has very low absorption in the IR region.

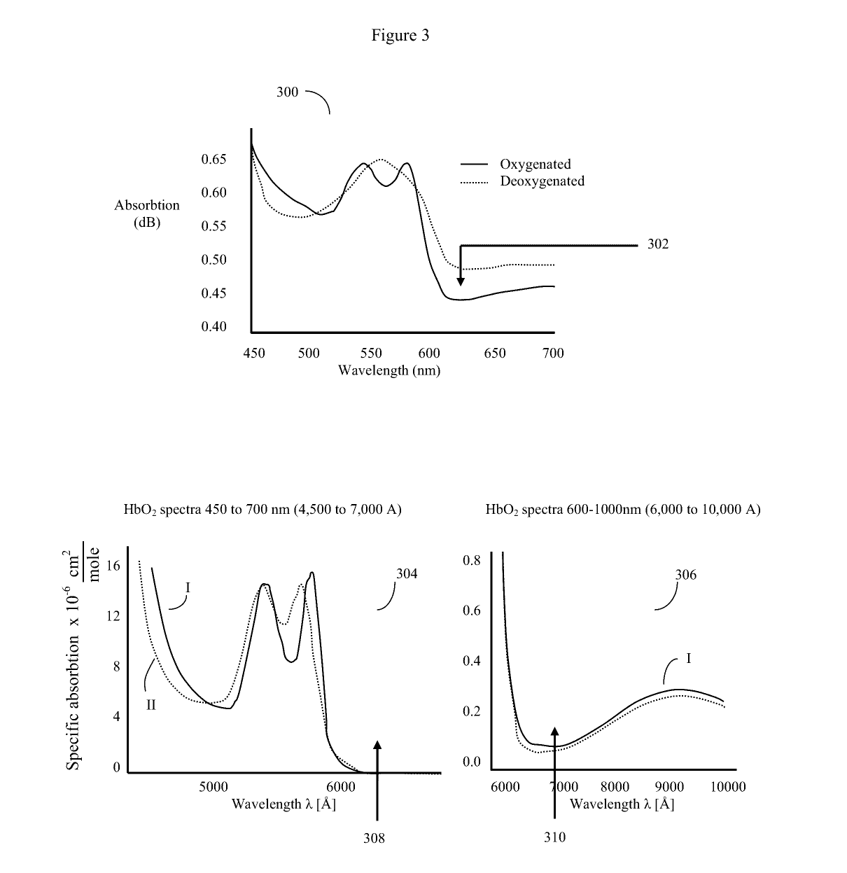

The spectrum of oxyhemoglobin is shown in FIG. 3.

Graph (300) is taken from the hemoglobin absorption spectra of Lang Quin Liu, et. al., Journal of Rehabilitation Research and Development, 43(4), July/August 2006, pages 553-564. This shows the spectra of oxy and deoxy hemoglobin in the 450 to 700 nm region. As is shown by (302), illumination light below about 600 nm penetrates far into the body because it is not absorbed by hemoglobin (the main light absorbing component in the body).

Graphs (304) and (306) are taken from Horecker, “The absorption spectra of Hemoglobin and its derivatives in the Visible and Near Infra-Red Regions”, Journal of Biological Chemistry, 1943, page 177. (304) shows the oxyhemoglobin spectra from about 425 nm to about 700 nm, and (306) shows the spectra from about 800 nm to 1000 nm. (Due to the age of the graph, the actual units are in Angstroms, where 10 angstroms=1 nm).

As (308) and (310) also show, illumination light in 600-800 nm regions (or alternatively the 650-800 nm region to avoid the region between 600 and 650 where residual tissue absorption is still present) penetrates far into the body because it is not absorbed by hemoglobin (the main light absorbing component in the body). Although the 600-800 nm region will thus be given in this disclosure as an example of a potentially preferred set of wavelengths useful for deep body penetration, it should be understood that this example is not intended to be limiting, and indeed inspection of FIG. 3 will show that wavelengths longer than 800 nm may also be used, because these wavelengths still penetrate far further into tissue than wavelengths less than 600 nm.

As a result, as previously discussed, the IR illumination light source may be located outside the heart and potentially even outside the body. The IR light source will penetrate past the near-transparent hemoglobin, and hit the upconverting material on the sides of the stent. The upconverting material will then emit UV light.

For example, Vivian Heyward and Dale Wagner, in “Applied body composition assessment”, 2^{nd} edition (2004), page 100 teach that Near Infrared light can penetrate the tissues for distances of up to 4 centimeters. Certain equations useful for calculating light penetration in tissue as a function of wavelength are given by Khalil et. al. in U.S. Pat. No. 7,043,287, the contents of which are incorporated herein by reference. Similarly in Ella, in US patent application 20070027411 [0014] teach that near infrared Low Level Laser Treatment light penetrates the body to a depth between 3-5 cm.

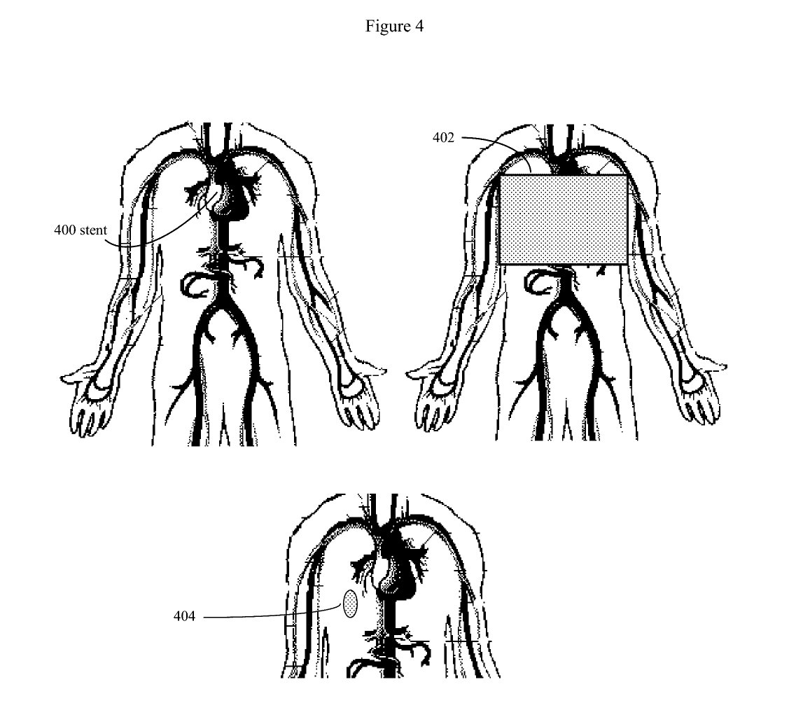

A diagram of the heart and body, showing various placement possibilities for the illumination light source, is shown in FIG. 4.

FIG. 4 (400) shows showing an upconverting stent implanted in a coronary artery. Here FIG. 4 (402) shows an example of an IR light source outside of the body—For example, a body wrap with a large (hundreds or thousands) number of IR LED diodes. Alternatively other light sources, such as one or more infrared lasers, may also be used. As will be discussed, light sources embedded into the medical device itself may be used as well.

FIG. 4 (404) shows an alternative example, where the IR light source could be applied from inside the body, either through an endoscope, alternatively through actually implanting IR light sources (e.g. IR LED's or fiber optics), or other means.

One way to do this is to produce a medical implant consisting of one or more infrared diodes or infrared lasers near the site of interest, potentially run an induction coil or other wireless means of powering the device to near the surface of the body, for example under the skin, and then power the infrared medical implant by supplying power to the induction coil. Here wireless power is applied to the induction coil or other wireless power receiver implanted under the patient's skin. Wires from the receiver transmit power to the infrared light producing implant, and the implant in turn supplies infrared light to the upconverting implant, typically located within 4-5 cm or less from the infrared light producing implant.

Given that the depth of infrared penetration into tissue is about 4-5 cm, other schemes to administer infrared light may also be used. This may include temporary insertion of optical catheters or endoscopes, or other means.

In alternative embodiments, the light source(s) may be made an integral part of the medical implant device itself. Here, for example, the medical implant may further comprise one or more red or infrared light sources (such as LEDs—light emitting diodes), and a power pickup, which can be an induction coil connected to the light source(s). In such arrangements, application of rapidly varying magnetic or electromagnetic fields to the induction coil can in turn provide power to the medical device embedded light emitting diodes, which in turn can result in cytotoxic chemicals being produced whenever the rapidly varying magnetic or electromagnetic fields are applied.

For indwelling catheters, depth of infrared light penetration will typically be less of a problem because many infections occur close to the surface of the body, less than 4-5 cm deep.

In some embodiments, the cytotoxic effects of the active material may be further enhanced by administering various types of UV activated or photocatalytic activated cytotoxic drugs, such as psorlens. In this discussion, we will assume that many of these UV activated drugs may be also photocatalytically activated as well.

High solubility psoralen compounds were taught by Hearst et. al., U.S. Pat. No. 4,169,204. Psorlens kill cells by cross-linking DNA strands when the psorlens are illuminated by UV light.

Bisaccia et. al., in U.S. Pat. No. 5,284,869, the contents of which are incorporated herein by reference, taught that the occurrence of restenosis following percutaneous transluminal coronary angioplasty is prevented or inhibited using a photopheresis treatment method. In accordance with the photopheresis treatment method, a photoactive compound such as 8-methoxypsoralen is administered to the patient's blood or affected tissue, or some fraction thereof, in vitro or in vivo using conventional administration routes. A portion of the patient's blood or affected tissue is then treated (preferably, extracorporeally) using photopheresis, which comprises subjecting the blood or affected tissue to electromagnetic radiation in a wavelength suitable for activating the photoactive compound, such as ultraviolet light, preferably long wavelength ultraviolet light in the wavelength range of 320 to 400 nm, commonly called UVA light. The treated blood or affected tissue, or a fraction thereof, is returned to the patient (in the case of extracoporeal photopheresis) or remains in the patient (following in vivo photopheresis).

Van Tassel et. al. in U.S. Pat. No. 6,719,778 taught that photoactivatable agents can also be used such that the energy activates the photoactivatable agent to cause a thickening of the vessel wall. For example, ultra-violet radiation can be used alone or in conjunction with a photoactivatable agent, such as a psoralen compound, to increase the adventitial volume of a blood vessel. Upon exposure to radiation, preferably ultra-violet-A radiation, the photoactivatable agent becomes activated and causes compositional and/or structural changes in the adventitia. However most stent problems are caused by unwanted tissue hyperplasia occurring in the stent area.

Various photoactivatable compounds are taught by Robinson et. al. U.S. Pat. No. 6,008,211, the contents of which are incorporated herein by reference.

Robinson teaches a broad class of photosensitive compounds having enhanced in vivo target tissue selectivity and versatility in photodynamic therapy. Many furocoumarin compounds, such as psoralens, exhibit cytostatic activity when photoactivated but exhibit little in vivo specificity for selectively accumulating in any particular target tissue such as atheromatous plaques. Reactive Oxygen Producing Photosensitizers (“ROPPs”) are photoactivatable compounds having an affinity for hyperproliferating cells (such as atheromatous plaque cells), which when photoactivated, produce cytotoxic reaction products. The photoactivity of a ROPP, such as a porphyrin, may be reduced by metalating the porphyrin while the selective affinity of the metalized ROPP for hyperproliferating tissue remains substantially unchanged. By linking a furocoumarin compound to a ROPP to form a F-ROPP, the cytostatic properties of the furocoumarin portion of the F-ROPP can be exploited while the selective affinity of the ROPP portion of the compound for hyperproliferating cells such as atheromatous plaque provides enhanced tissue selectivity without cytotoxicity. In vivo, certain F-ROPPs may be forced to selectively accumulate in a target tissue by illuminating only the target tissue with light having a wavelength operable for photoactivating the F portion of the F-ROPP thereby causing the F-ROPP to either form a monoadduct with or crosslink the cellular DNA in the target tissue. Light of a second wavelength can then be delivered to the target tissue to photoactivate the ROPP portion causing further interference with cellular activity.

Robinson's table of potentially useful UV activated drugs (his Table I) includes Pyrrole-derived macrocyclic Texaphyrins and derivatives compounds thereof (11) Naturally occurring or synthetic Phenoxazine dyes and derivatives porphyrins and derivatives thereof (12) thereof (1) Phenothiazines and derivatives Naturally occurring or synthetic thereof (13) chlorins and derivatives thereof (2) Chalcoorganapyrylium dyes and Naturally occurring or synthetic derivatives thereof (14) bacteriochlorins and derivatives Triarylmethanes and derivatives thereof (3) thereof (15) Synthetic isobacteriochlorins and Rhodamines and derivatives derivatives thereof (4) thereof (16) Phthalocyanines and derivatives Fluorescenes and derivatives thereof (5) thereof (17) Naphthalocyanines and derivatives Azaporphyrins and derivatives thereof (6) thereof (18) Porphycenes and derivatives Benzochlorins and derivatives thereof (7) thereof (19) Porphycyanines and derivatives Purpurins and derivatives thereof (8) thereof (20) Pentaphyrin and derivatives Chlorophylls and derivatives thereof (9) thereof (21) Sapphyrins and derivatives Verdins and derivatives thereof (22) thereof (10)

Other compositions were also taught by Robinson et. al., U.S. Pat. No. 5,773,609, incorporated herein by reference.

Returning to active material, photocatalytic particles, such as Titanium Dioxide (TiO₂) can be photoactivated to a form that can kill microorganisms (i.e. bacteria, fungi). Although previously, use of such photoactivated antimicrobial agents for some applications was hampered by the difficulty of providing short wavelength photoactivation light to medical devices implanted in the body, such as catheters, use of the invention's active materials solves this problem.

Medical devices employing photoactivated antimicrobial agents may be constructed using the active material, and then illuminated with longer wavelength light, such as light in the 600-800 nm region, that penetrates the body well. The upconverting material on the active material portion of the medical device will convert the longer wavelength light into shorter wavelength light capable of photoactivating the photoactivated anti-microbial agent. The photoactivated anti-microbial agent can, in turn, destroy bacteria, fungi, or other microbes near the implanted medical device.

Alternatively the active material can provide a photoactivated agent that can be used to exert a cytotoxic effect against the patient's cells as well. As previously discussed, a stent composed of a mixture of upconverting material particles and photoactivated particles (again using TiO₂ particles as an example), generally held in a biocompatible carrier or support material, will destroy unwanted human cells. These unwanted human cells can be proliferating cells causing unwanted restenosis. Such cells are killed by cytotoxic chemicals, such as free radicals, produced by the photoactivated particles. Again, as previously discussed, the mixture of upconverting particles, photoactivated particles, and a biocompatible particle support material is here termed “active material”.

Again, it should be understood that although the stent examples and the catheter examples have been used as specific examples of an implantable medical device incorporating the active material disclosed herein, other implantable medical devices may also be improved by the incorporation of the active material.

Examples of other applications include implantable active material “seeds” for treatment of malignant or non-malignant growths or cancer. Here often one or more (i.e. a plurality) of pellets or seeds comprised of active material will be implanted in the region where cell death is desired. Cell death will be mediated by exposing the active material containing pellets or seeds to IR light, thus producing cytotoxic chemicals. This effect may be further enhanced, as desired, by also using other photocatalytic or light activated cytotoxic drugs as desired.

In order to ensure that the active material seeds can be retrieved from the body when their use is no longer desired, it may be useful to additionally incorporate these seeds into x-ray opaque or MRI contrasting carriers so that the location of the seeds can be more easily identified, or else dope the seeds with additional x-ray opaque or MRI contrast material as needed.

In alternative embodiments, an additional photocatalytic activated drug may also be used that is not directly cytotoxic, but may be light activated to a form that causes the drug to act as a signal to other drugs or drug carrier moieties, informing the moieties that cells in the region of the photocatalytically activated drug should be targeted for various forms of chemical therapy or biologic therapy.

For example, the photocatalytic activity of the device could convert a first apo-target molecule from a form that does not serve as a target for another receptor molecule (such as an antibody or protein) to a form (target-molecule) that now does serve as a target for the other receptor molecule. Examples of such molecules that can be converted from a first apo-target form to a second target-form include various light receptor molecules (i.e. retinal, rhodopsin, G-proteins, UV light receptor complexes, various light receptor proteins, LOV domain proteins, and the like).

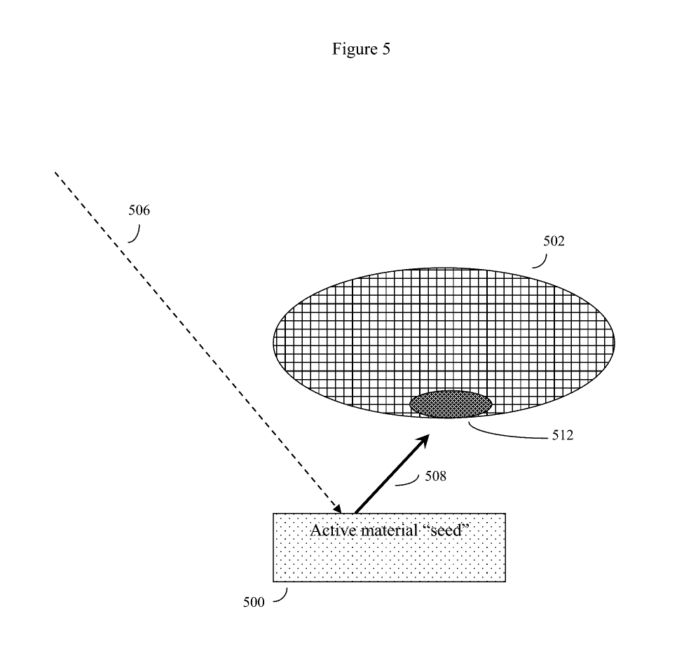

FIG. 5 shows an example of how an active material containing implant, such as implantable “seeds”, may produce a desired therapeutic treatment. In FIG. 5 (500), an active material containing implant “seed” is implanted in or near a diseased portion of the body, such as a tumor (502).

When exposed to longer wavelength excitation light (506), the active material “seed” (500) emits cytotoxic chemicals (508). These cytotoxic chemicals (508) turn damage a nearby portion of the tumor (512).

Although, as previously discussed, in many embodiments, the active material will generally be used in macroscopic, fixed position, implantable medical devices with largest dimensions significantly greater than 1 millimeter, such as 1 centimeter or longer are contemplated. In other embodiments, smaller devices may also be implemented.

In other embodiments, the implantable medical device may be comprised of fractional millimeter sized particles of active material that can have their surface properties modified to make them water soluble, fat soluble, insoluble, or bind to specific organs or locations of the body (for example by binding specific antibodies, receptors, ligands, or targets to the particles).

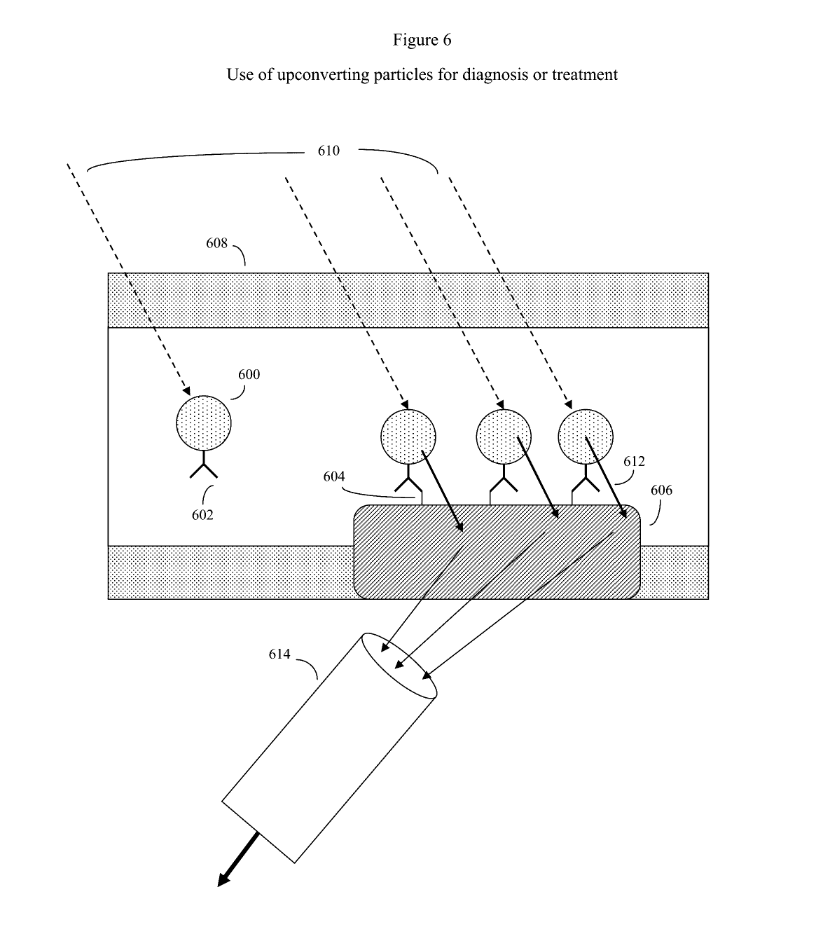

FIG. 6 shows an example of how small (greatest dimension less than one millimeter) particles of active material might be used for a medical diagnostic procedure. In this example, the active material particles (600) are first bound to an antibody (602) against a molecular target (604) on a tissue or cell (often a diseased tissue or cell) of interest (606). The particles are then introduced into a body lumen (608), which could be into the blood stream, into the gastrointestinal tract, into the lung, into urinary tract, or other area. The active material particles will tend to preferentially bind to the tissue or cells that express the target (606).

In this diagram, the body lumen is an artery or vein, and the upconverting particles small enough to be carried along by the flow of the blood circulation.

When illuminated with longer wavelength light (610), the active material particles may be used to therapeutically treat the tissue or cells by delivering cytotoxic chemicals directly to the affected area.

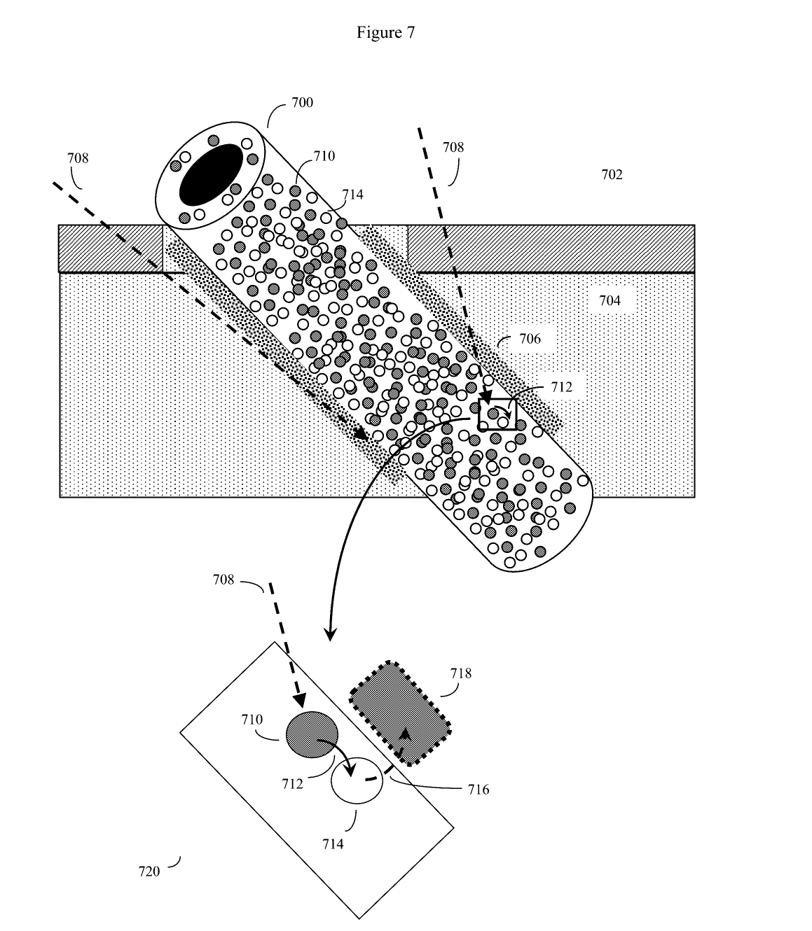

FIG. 7 shows a medical catheter, such as a Broviac® catheter, or other catheter, that spans the space between outside of the patient's body and inside the patient's body, and thus poses a considerable risk of contamination by various microorganisms at the interface, and subsequent infection.

Here, risk of infection can be reduced by constructing at least certain portions of the catheter with active material comprising upconverting particles, as well as particles of a photocatalytic antibacterial material, such as particles of titanium dioxide.

In some embodiments, these particles of upconverting material and photocatalytic material may be dispersed in the support material so as to form a compound or aggregate where the various particles are mixed into the body of the support material, thus forming an entire section of catheter made from active material.

Alternatively the active material may itself be dispersed as a wrap, outer layer, or surface coating on the exterior surface of the catheter, at least near those regions of the catheter where anti-microbial action is desired.

FIG. 7 (700) shows a portion of such a catheter, spanning the region from outside the patient's body (702) to inside the patient's body (704). In this example, there is an infection at the junction, and thus the catheter (700) is surrounded by a biofilm (706) undesired microbes such as pathogenic bacteria.

These microbes can be destroyed by illuminating the catheter with 600-800 nm light (708), which penetrates deeply into the body. The upconverting particles (710) in the catheter's active material produce shorter wavelength light (712), which activates the photocatalytic particles (714), thus producing cytotoxic antibacterial material (716). The cytotoxic chemicals (716) in turn kill the various undesired pathogenic bacteria in the biofilm (718). A higher magnification view of this process is shown in (720).

Various types of photocatalytic antimicrobial materials may be used. These materials include Na-Pheophorbide, semiconductor particles, particles such as Titanium Dioxide (TiO₂), as well as various forms of TiO₂ such as nitrogen- and carbon-doped TiO₂ substrates, and other suitable photoactivated antimicrobial or cytotoxic agents. Such agents may include various doped and non-doped forms of TiO₂, NaTaO₃, ZnO, CdS, GaP, SiC, WO₃, ZnS, CdSe, SrTiO₃, CaTiO₃, KTaO₃, Ta₂O₅ ZrO₂, and so on. In some embodiments, it may be useful to enhance the antimicrobial activity of the upconverting medical device by also using other agents, such as Ag (silver) nanoparticles, to increase the efficiency of the antimicrobial or cytotoxic process.

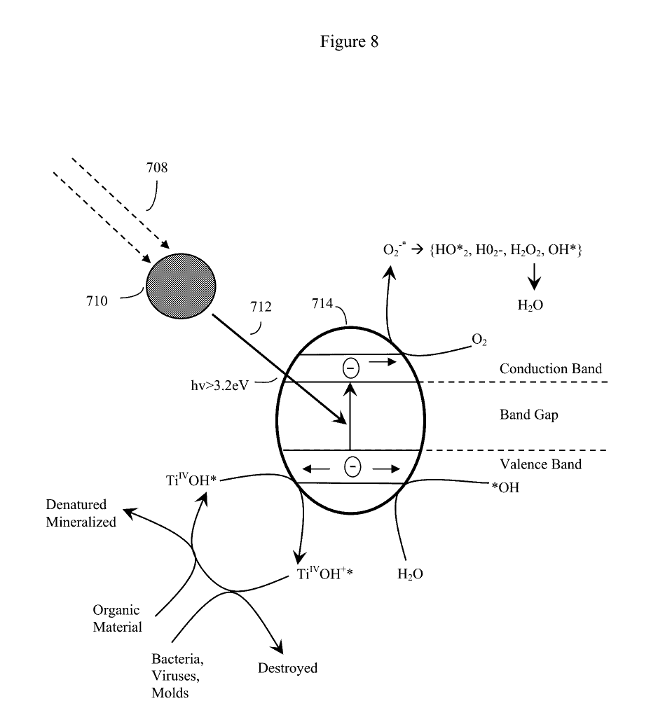

One proposed mechanism for this cytotoxic (antimicrobial) effect, taken from FIG. 15 of Francis (US patent application 2008/0039768) is shown in FIG. 8. FIG. 8 shows a detail of some of the reactions that may be going on inside of the photoactivated catalyst (714) previously shown in FIG. 7. As before, longer wavelength infrared light (708) is absorbed by upconverting particle or material (710), which emits a shorter wavelength light (712) which in turn is absorbed by, and activates, photoactivated catalyst (714).

This approach offers a number of advantages over prior art approaches. For example, Francis, in US patent application 2008/0039768, incorporated herein by reference, taught a medical device such as a catheter with a photocatalytic layer with an antimicrobial material, such a doped semiconductor oxide (e.g. TiO₂ and other compounds), which could be illuminated by electromagnetic radiation. However because Francis could not determine how to easily send activating light to his photocatalytic compounds, he had to devise complex illumination methods such as fiber optic cables, wave guides, light ports, UV transmissible wave guides, LEDs, antenna, and the like to convey the photoactivating light to the surface of the catheter. By contrast, by also including upconverting materials into the catheter, the catheter may be simply illuminated by infrared light, in particular 600-800 nm light which penetrates deeply into tissues, from an outside source. Thus the catheter design itself may be simplified, because the light source can now be entirely outside the body if this is desired.

Note that this same approach may be used for other implantable devices, such as stents, seeds, etc. That is, the activated semiconducting material has a general destructive effect on organic material. Although this destructive effect may be utilized to destroy bacteria, viruses, and molds, it may also be used to destroy unwanted tissue, such as unwanted tissue acting to close an implanted stent. Thus by making medical implants, such as stents and seeds, out of a combination of an upconverting material and a photoactivated catalyst, such as TiO₂, other semiconductor material (previously discussed) or other reusable photoactivated catalyst, the implant (such as the stent) may be implanted, and then used for years, retaining its cytotoxic capability whenever illuminated with sufficient quantities of infrared light.

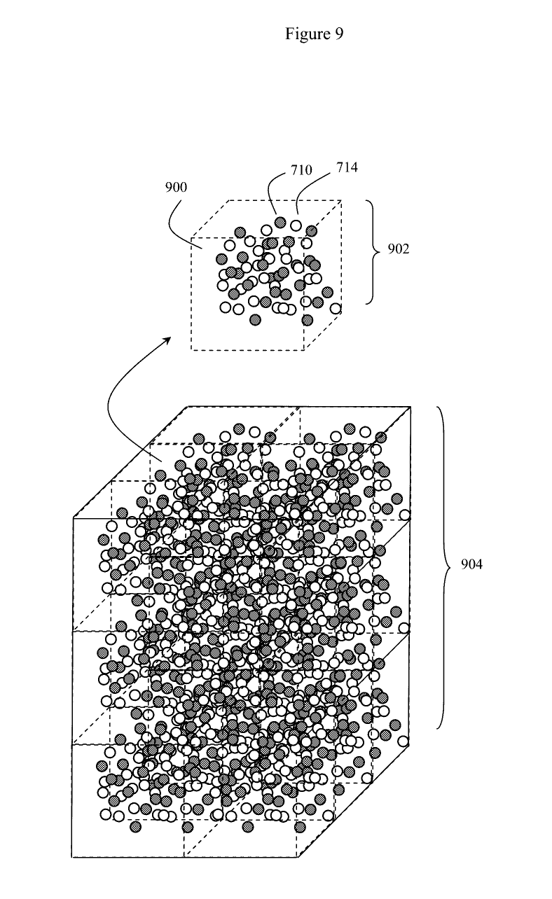

FIG. 9 shows a close up of active material. This active material comprises a mixture, compound, or aggregate material of various upconverting and photocatalytic particles (710, 714) dispersed inside of non-toxic, non-soluble biocompatible particle support material (900), thus forming various sizes of active material (902, 904).

In a preferred embodiment, this support material (900) conducts at least infrared light so that the upconverting particles (910) in the interior of the active material are suitably energized (here the efficiency will be greater if the support material also is reasonably transmissive to blue and ultraviolet light as well). This support material (900) should preferably be at least porous to small materials, such as gasses and water molecules, so that the cytotoxic chemicals generated by the various photocatalytic particles (914) inside of the active material can exit the active material.

This active material may be made in various ways. If the support material is a polymer, a mixture of the various photocatalytic particles and upconverting particles may be mixed with the monomer (or lower polymerization state) form of the polymer, and further polymerization then induced or allowed to occur, thus trapping the various particles in the interior of the polymer. Alternatively, if the support material has a melting point that is higher than body temperature, but lower than the temperature that destroys the structure of the photocatalytic particles and upconverting particles, then the support material may be heated above its melting point, the various particles added, and the support material then allowed to cool, again trapping the various particles in the interior of the support material.

The permeability of the support material to molecules of various sizes can vary. Generally the support material will have a porous structure that on the low side is at least permeable to gas and optionally also water molecules, but that on the high side is impermeable to the various particles (otherwise the upconverting particles and the photocatalytic particles will eventually diffuse out of the support material). Thus if very small (2-10 nm) upconverting quantum dots are used, the pore size of the support material may have to be in the 1 nanometer range. By contrast, if both the upconverting material and the catalytic material are larger sized particles, such as pigment grade TiO₂ particles with sizes in the 200 nanometer range, then the pore size of the support material may be larger, such as in the 100+ nanometer size ranges.

Drug Use Examples:

In one treatment mode, a patient is administered UV activated or photocatalytically activated cytotoxic drugs, and kept away from other UV light sources. The upconverting stent (or other active material containing medical device) is illuminated by infrared (IR) light. The stent (or other medical device) produces cytotoxic chemicals exactly where it is needed—that is, local to the stent (or other device) where the chances of unwanted cell proliferation and hyperplasia is greatest. The UV activated cytotoxic drugs are carried to the stent area, where they interact with the UV light emitted by the stent, and kill the unwanted proliferating cells.

One advantage of this approach that the cytotoxic effect of the stent can have a very long lifetime. Unlike drug eluting stents, where the stent eventually runs out of the drug, an IR to UV upconverting stent could have a potential lifetime of years or more, because it never runs out of material. That is, the upconverting material can be made of a glass or ceramic or other long-life material, and produce UV photons whenever illuminated with outside light.

Use with Drug Pumps

In some cases, the efficiency and selectivity of drug administration may be further enhanced by delivering the light activated or photocatalytically activated drug to an area of the body local or “upstream” (from a circulatory sense) from the implanted upconverting medical device. In this manner, drug selectivity is enhanced both by the local acting nature of the active material in the implanted upconverting medical device, and by the localized nature of the drug administration pump.

Other Concepts:

In an alternative embodiment, as previously discussed, the stent or other medical device itself could contain a plurality of UV light emitting diodes, and suitable energy harvesting circuitry to power the UV light emitting diodes from an external radiofrequency source.

In other alternative embodiments, holes in the stent or other medical device may contain one more sensing elements. These sensing elements would be used to determine if undesired cell hyperplasia had occurred. Examples of such sensing elements can include a flexible membrane placed over one or more small holes in the stent. The flexible membrane may contain magnetic material and/or electrically conducting material. When exposed to an oscillating magnetic material, the flexible membrane will vibrate at a first set of frequencies if the membrane is not covered with overgrown cells, but will vibrate at a second set of frequencies if the membrane is not covered with the cells. This difference in vibration frequencies could be detected by suitable magnetic or radiofrequency pickups, and may be used to diagnose the status of the stent prior to treatment.

In yet another alternative environment, holes in the stent or other medical device might contain magnetic fibers or filaments that can be induced to move by external magnetic fields, and be used to actively clean portions of the stent area.

1. An implantable medical device comprising:
at least one non-soluble active material formed from upconverting particles and photocatalytic particles embedded inside at least one different non-toxic, non-soluble biocompatible particle support material;
wherein when said active material is exposed to red or infrared light, said upconverting particles emit shorter wavelength light which is subsequently absorbed by said photocatalytic particles, which in turn emit cytotoxic chemicals in response to said shorter wavelength light;
wherein said device remains where originally implanted;
wherein said device operates by localized destruction, relative to a location of said device, of unwanted living cells surrounding said active material, said localized destruction caused by cytotoxic chemicals emitted by said active material in response to an interaction of red or infrared light with said active material.

2. The implantable medical device of claim 1, in which particle support material conducts at least infrared light, and is permeable to at least said cytotoxic chemicals but not permeable to either said upconverting particles or said photocatalytic particles.

3. The device of claim 1, in which said upconverting particles absorb infrared light, and emit visible or ultraviolet light.

4. The device of claim 3, in which said red or infrared light comprises light of wavelengths longer than 600 nm; and
said shorter wavelength light comprises light of wavelengths shorter than 500 nm.

5. The device of claim 1, in which said photocatalytic particles are semiconductor particles.

6. The device of claim 1, in which said photocatalytic particles are titanium dioxide particles.

7. The implantable medical device of claim 1, wherein said at least one different non-toxic, non-soluble, biocompatible particle support material has a greatest dimension of 1 millimeter or longer.

8. The implantable medical device of claim 1, wherein said device is configured to retain its cytotoxic capability, when illuminated with infrared light, for time periods of a year or greater.

9. An implantable medical device comprising:
at least one non-soluble active material formed from upconverting particles photocatalytic particles embedded inside at least one different non-toxic, non-soluble biocompatible particle support material;
said device further comprising a non-soluble biocompatible active material support material disposed to hold said active material;
wherein when said active material is exposed to red or infrared light, said upconverting particles emit shorter wavelength light which is subsequently absorbed by said photocatalytic particles, which in turn emit cytotoxic chemicals in response to said shorter wavelength light;
in which said device further comprises a device shape;
wherein said device's shape is determined by any of said shape of said active material, and said active material support materials disposed to hold said active material;
wherein said device remains where originally implanted;
wherein said device operates by localized destruction, relative to a location of said device, of unwanted living cells surrounding said active material, said localized destruction caused by cytotoxic chemicals emitted by said active material in response to an interaction of red or infrared light with said active material.

10. The implantable medical device of claim 1, in which particle support material conducts at least infrared light, and is at least permeable to said cytotoxic chemicals but not permeable to either said upconverting particles or said photocatalytic particles.

11. The device of claim 9, in which said upconverting particles absorb infrared light, and emit visible or ultraviolet light, and said photocatalytic particles are activated by said visible or ultraviolet light.

12. The device of claim 9, in which said red or infrared light comprises light of wavelengths longer than 600 nm; and
said shorter wavelength light comprises light of wavelengths shorter than 500 nm.

13. The device of claim 9, in which said device, when illuminated by photons at a first longer wavelength emits photons at a second shorter wavelength, and in which said photons at said second shorter wavelength activate said photocatalytic particles, and said photocatalytic particles have direct cytotoxic effects against human cells or microbes surrounding said active material.

14. The implantable medical device of claim 9, wherein said at least one different non-toxic, non-soluble biocompatible particle support material comprises at least one of plastic, silicone, glass, or ceramic material; and said non-soluble biocompatible support material disposed to hold said active material comprises at least one of a metal, nitinol, plastic, silicone, glass, or ceramic material.

15. The device of claim 9, in which said photocatalytic particles are semiconductor particles.

16. The device of claim 15, in which said photocatalytic particles are titanium dioxide particles.

17. The implantable medical device of claim 9, wherein said device is configured to retain its cytotoxic capability, when illuminated with infrared light, for time periods of a year or greater.

18. A macroscopic material comprising:
at least one non-soluble active material with a greatest dimension of at least 1 millimeter; said active material comprising upconverting particles and photocatalytic particles embedded inside least one different non-toxic, non-soluble, biocompatible particle support material that is at least permeable to gas but not permeable to either said upconverting particles or said photocatalytic particles;
wherein when said active material is exposed to red or infrared light, said upconverting particles emit shorter wavelength light which is subsequently absorbed by said photocatalytic particles, which in turn emit cytotoxic chemicals or perform other chemical reactions in response to said shorter wavelength light.

19. The material of claim 18, wherein said material is substantially solid and rigid enough to substantially maintain its shape in an aqueous environment at around 37° Centigrade.
